(19) United States
(12) Patent Application Publication
(10) Pub. No.: US 2010/0322376 A1
(43) Pub. Date: Dec. 23, 2010

(54) RADIATION IMAGING APPARATUS AND METHOD

(75) Inventors: Tomonari SENDAI, Ashigarakami-gun (JP); Yasuko YAHIRO, Ashigarakami-gun (JP); Makoto SUGIZAKI, Ashigarakami-gun (JP); Naoto IWAKIRI, Ashigarakami-gun (JP); Sadato AKAHORI, Ashigarakami-gun (JP); Yasunori OHTA, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/819,315

(22) Filed: Jun. 21, 2010

(30) Foreign Application Priority Data
Jun. 22, 2009 (JP) 2009-147267

(51) Int. Cl. A61B 6/03

(52) U.S. Cl. 378/8

(57) ABSTRACT

A plurality of radiation images of an object are acquired by iterating the steps of: beginning driving of a rotation driving unit at the time at which a breathing signal having been detected by a breathing sensor has come into a predetermined state, performing a radiation imaging operation with a set of a radiation source and a radiation detector at the time, at which the heart has come into a predetermined state, in accordance with a heartbeat signal having been detected by a heartbeat sensor within a rotation driving period, during which the rotation driving unit performs the rotation driving, and ceasing the rotation driving of the rotation driving unit at the time at which the breathing of the object has come into a state other than the predetermined state.

### BACKGROUND OF THE INVENTION

1. Field of the Invention

This invention relates to a radiation imaging apparatus and a radiation imaging method, wherein radiation imaging operations are performed by rotating a set of a radiation source and a radiation detector with respect to an object.

2. Description of the Related Art

Recently, radiation imaging techniques are employed, wherein a plurality of radiation images of an object are acquired by performing radiation imaging operations on the object from a plurality of different angles by use of CT scanners, or the like, and wherein a three-dimensional image (volume data) is formed by reconstructing the thus acquired radiation images. In such cases, it often occurs that a state of the object, such as the state of the heart, varies among the plurality of the radiation images, which have been recorded from the different angles, due to heartbeat and breathing, which are the physiological phenomena of persons. The variation in the state of the object adversely affects image quality of the three-dimensional image.

Therefore, it has heretofore been proposed to perform radiation imaging operations in accordance with a period, with which the breathing is made, and the heartbeat. (Reference may be made to, for example, Japanese Unexamined Patent Publication Nos. 2000-262513 and 2005-021345.) Japanese Unexamined Patent Publication No. 2000-262513 discloses a radiation imaging technique, wherein the radiation imaging operations are performed a plurality of times in a manner synchronized with the breathing and the heartbeat, wherein the occurrence of a shift of a pattern of the heart, or the like, among the plurality of the radiation images is thereby prevented, and wherein the radiation imaging operations are thus performed quickly.

In cases where the period, with which the rotation of a set of an X-ray tube and a radiation detector is made, is short as in the cases of the apparatuses described in Japanese Unexamined Patent Publication Nos. 2000-262513 and 2005-021345, and in cases where the plurality of the radiation images per turn are detected within approximately 0.5 second, the patient need not cease the breathing forcibly, and it is possible to acquire the plurality of the radiation images at the time of the state of the heart in a predetermined temporal phase. However, in the cases of a cone-beam CT scanner, in which a set of a radiation source and a radiation detecting unit is rotated one turn around an object, a period of time ranging from approximately several seconds to approximately several tens of seconds is necessary for the detection of a plurality of radiation images. Therefore, in cases where the radiation imaging operations are to be performed by the CT scanner so as to match with the heartbeat period and the breathing period, there is the problems in that the necessary number of the radiation images are not capable of being acquired during one time of the breathing period, and in that it is not always possible for the CT scanner, or the like, to perform the radiation imaging operations so as to match with the heartbeat period and the breathing period.

### SUMMARY OF THE INVENTION

The primary object of the present invention is to provide a radiation imaging apparatus, wherein a plurality of radiation imaging operations in accordance with heartbeat and breathing for suppressing occurrence of an artifact are performed efficiently. Another object of the present invention is to provide a radiation imaging method, wherein a plurality of radiation imaging operations in accordance with heartbeat and breathing for suppressing occurrence of an artifact are performed efficiently.

The present invention provides a radiation imaging apparatus, comprising:

i) a radiation source for irradiating radiation to an object,

ii) a radiation detector for detecting the radiation carrying image information of the object at the time at which the radiation is irradiated from the radiation source to the object,

iii) a rotation driving unit for rotating the set of the radiation source and the radiation detector around the object,

iv) a breathing sensor for detecting a state of breathing of the object as a breathing signal,

v) a heartbeat sensor for detecting a state of heartbeat of the object as a heartbeat signal,

vi) rotation control means for controlling such that the driving of the rotation driving unit is begun at the time at which the breathing signal having been detected by the breathing sensor has come into a predetermined state, and such that the driving of the rotation driving unit is ceased at the time at which the breathing of the object has come into a state other than the predetermined state, and

vii) imaging operation control means for controlling the operation of the set of the radiation source and the radiation detector, such that a radiation imaging operation is performed at the time, at which the heart has come into a predetermined state, in accordance with the heartbeat signal having been detected by the heartbeat sensor within a rotation driving period, during which the rotation driving unit performs the rotation driving by being controlled by the rotation control means.

The present invention also provides a radiation imaging method, utilizing:

i) a radiation source for irradiating radiation to an object,

ii) a radiation detector for detecting the radiation carrying image information of the object at the time at which the radiation is irradiated from the radiation source to the object,

iii) a rotation driving unit for rotating the set of the radiation source and the radiation detector around the object,

iv) a breathing sensor for detecting a state of breathing of the object as a breathing signal, and

v) a heartbeat sensor for detecting a state of heartbeat of the object as a heartbeat signal,

wherein a plurality of radiation images of the object are acquired by iterating the steps of:

beginning the driving of the rotation driving unit at the time at which the breathing signal having been detected by the breathing sensor has come into a predetermined state,

performing a radiation imaging operation with the set of the radiation source and the radiation detector at the time, at which the heart has come into a predetermined state, in accordance with the heartbeat signal having been detected by the heartbeat sensor within a rotation driving period, during which the rotation driving unit performs the rotation driving, and

ceasing the rotation driving of the rotation driving unit at the time at which the breathing of the object has come into a state other than the predetermined state.

The predetermined state of the breathing signal may be the state, in which the breathing is stable, and should preferably be the state, in which the breathing signal represents, for example, at least 95% of the maximum inspiration. Also, the predetermined state of the heart may be the state, in which the heartbeat signal represents approximately an identical temporal phase among the plurality of the radiation imaging operations.

The rotation driving unit may rotate the set of the radiation source and the radiation detector at a predetermined velocity. In such cases, the imaging operation control means may control the operation of the set of the radiation source and the radiation detector, such that the radiation imaging operation is performed after the set of the radiation source and the radiation detector has been driven for rotation from the state, in which the set of the radiation source and the radiation detector stops, and after the velocity of the set of the radiation source and the radiation detector has thus come up to the predetermined velocity.

In cases where the radiation imaging operation is performed after the velocity of the set of the radiation source and the radiation detector has come up to the predetermined velocity, the rotation control means may have a function for returning the position of the set of the radiation source and the radiation detector in the direction, which is reverse to the direction of the rotation of the set of the radiation source and the radiation detector at the time of the radiation imaging operation, after a radiation imaging operation performed most recently has been completed or before a next radiation imaging operation is begun, such that the rotation driving of the set of the radiation source and the radiation detector is begun at the predetermined velocity from a predetermined position. Particularly, in such cases, the rotation control means may have the function for returning the position of the set of the radiation source and the radiation detector, such that the rotation driving of the set of the radiation source and the radiation detector is begun at the predetermined velocity from the position, at which the rotation driving of the rotation driving unit has been ceased at the stage of the radiation imaging operation performed most recently.

Alternatively, the imaging operation control means may control such that the radiation imaging operation is performed at the time, at which the set of the radiation source and the radiation detector takes a predetermined angular position, during the period between the state, in which the set of the radiation source and the radiation detector stops, and the stage, at which the velocity of the set of the radiation source and the radiation detector comes up to the predetermined velocity.

As another alternative, the rotation control means may control the rotation driving unit such that the set of the radiation source and the radiation detector is rotated a plurality of turns. In such cases, the imaging operation control means may control the operation of the set of the radiation source and the radiation detector, such that the radiation imaging operation is performed with respect to an angular position taken by the set of the radiation source and the radiation detector during the period between the state, in which the set of the radiation source and the radiation detector stops at the time of a predetermined turn, and the stage, at which the velocity of the set of the radiation source and the radiation detector comes up to the predetermined velocity at the time of the predetermined turn, the radiation imaging operation with respect to the angular position being performed at the time of the next turn. The set of the radiation source and the radiation detector may be rotated the plurality of turns in a predetermined direction. Alternatively, the set of the radiation source and the radiation detector may be rotated the plurality of turns by being rotated in the predetermined direction and in the reverse direction.

Also, the radiation imaging operation may be performed with the predetermined imaging operation timing described above under the condition in which the set of the radiation source and the radiation detector is being rotated. Alternatively, the rotation control means may control such that the rotation driving of the set of the radiation source and the radiation detector by the rotation driving unit is ceased at the time of the radiation imaging operation, and such that the rotation driving is again begun after the radiation imaging operation has been performed.

Further, the imaging operation control means may control the operation of the set of the radiation source and the radiation detector, such that the radiation imaging operation is performed at the time, at which a heartbeat signal component representing that the heart has come into the predetermined state has been detected by the heartbeat sensor, within the rotation driving period, during which the rotation driving unit performs the rotation driving by being controlled by the rotation control means.

With the radiation imaging apparatus and the radiation imaging method in accordance with the present invention, the plurality of the radiation images of the object are acquired by iterating the steps of:

beginning the driving of the rotation driving unit at the time at which the breathing signal having been detected by the breathing sensor has come into the predetermined state,

performing the radiation imaging operation with the set of the radiation source and the radiation detector at the time, at which the heart has come into the predetermined state, in accordance with the heartbeat signal having been detected by the heartbeat sensor within the rotation driving period, during which the rotation driving unit performs the rotation driving, and

ceasing the rotation driving of the rotation driving unit at the time at which the breathing of the object has come into a state other than the predetermined state.

Thus, with the radiation imaging apparatus and the radiation imaging method in accordance with the present invention, in cases where the set of the radiation source and the radiation detector is rotated around the object and performs the radiation imaging operations in accordance with the breathing and the heartbeat of the object, the rotation driving is ceased with respect to the period, during which the breathing and the imaging operation timing do not match with each other, and during which the radiation imaging operation is not capable of being performed. Thus the problems are prevented from occurring in that the rotation driving alone is performed without the set of the radiation source and the radiation detector performing the radiation imaging operation. Therefore, the plurality of the radiation images are acquired efficiently, such that the adverse effects of variation in state of the object due to the breathing and the heartbeat of the object are suppressed to the minimum.

The radiation imaging apparatus in accordance with the present invention may be modified such that the rotation driving unit rotates the set of the radiation source and the radiation detector at the predetermined velocity, and

the imaging operation control means controls the operation of the set of the radiation source and the radiation detector, such that the radiation imaging operation is performed after the set of the radiation source and the radiation detector has been driven for rotation from the state, in which the set of the radiation source and the radiation detector stops, and after the velocity of the set of the radiation source and the radiation detector has thus come up to the predetermined velocity.

At the time at which the rotation of the set of the radiation source and the radiation detector is begun, a certain length of time is required due to inertia force before the velocity of the set of the radiation source and the radiation detector comes up to the predetermined velocity. With the modification described above, wherein the imaging operation control means controls the operation of the set of the radiation source and the radiation detector, such that the radiation imaging operation is begun after the velocity of the set of the radiation source and the radiation detector has come up to the predetermined velocity, a difference in imaging operation conditions among the plurality of the radiation imaging operations is suppressed to the minimum, and image quality of the three-dimensional image reconstructed from the plurality of the radiation images is prevented from becoming bad.

Also, the radiation imaging apparatus in accordance with the present invention may be modified such that the rotation control means has the function for returning the position of the set of the radiation source and the radiation detector in the direction, which is reverse to the direction of the rotation of the set of the radiation source and the radiation detector at the time of the radiation imaging operation, after the radiation imaging operation performed most recently has been completed or before the next radiation imaging operation is begun, such that the rotation driving of the set of the radiation source and the radiation detector is begun at the predetermined velocity from the predetermined position. Particularly, in such cases, the radiation imaging apparatus in accordance with the present invention may be modified such that the rotation control means has the function for returning the position of the set of the radiation source and the radiation detector, such that the rotation driving of the set of the radiation source and the radiation detector is begun at the predetermined velocity from the position, at which the rotation driving of the rotation driving unit has been ceased at the stage of the radiation imaging operation performed most recently. With the modification described above, the problems are prevented from occurring in that a position, at which the radiation imaging operation is not performed, arises with respect to the period between the stage, at which the rotation of the set of the radiation source and the radiation detector is begun, and the stage, at which the velocity of the set of the radiation source and the radiation detector comes up to the predetermined velocity.

Further, the radiation imaging apparatus in accordance with the present invention may be modified such that the imaging operation control means controls such that the radiation imaging operation is performed at the time, at which the set of the radiation source and the radiation detector takes the predetermined angular position, during the period between the state, in which the set of the radiation source and the radiation detector stops, and the stage, at which the velocity of the set of the radiation source and the radiation detector comes up to the predetermined velocity. With the modification described above, the problems are prevented from occurring in that a position, at which the radiation imaging operation is not performed, arises with respect to the period between the stage, at which the rotation of the set of the radiation source and the radiation detector is begun, and the stage, at which the velocity of the set of the radiation source and the radiation detector comes up to the predetermined velocity.

Furthermore, the radiation imaging apparatus in accordance with the present invention may be modified such that the rotation control means controls the rotation driving unit such that the set of the radiation source and the radiation detector is rotated the plurality of turns, and

the imaging operation control means controls the operation of the set of the radiation source and the radiation detector, such that the radiation imaging operation is performed with respect to the angular position taken by the set of the radiation source and the radiation detector during the period between the state, in which the set of the radiation source and the radiation detector stops at the time of the predetermined turn, and the stage, at which the velocity of the set of the radiation source and the radiation detector comes up to the predetermined velocity at the time of the predetermined turn, the radiation imaging operation with respect to the angular position being performed at the time of the next turn.

With the modification described above, the problems are prevented from occurring in that a position, at which the radiation imaging operation is not performed, arises with respect to the period between the stage, at which the rotation of the set of the radiation source and the radiation detector is begun, and the stage, at which the velocity of the set of the radiation source and the radiation detector comes up to the predetermined velocity.

Also, the radiation imaging apparatus in accordance with the present invention may be modified such that the rotation control means begins the driving of the rotation driving unit at the time, at which the breathing signal represents at least 95% of the maximum inspiration, and such that the rotation control means ceases the driving of the rotation driving unit at the time, at which the breathing signal represents a level lower than 95% of the maximum inspiration. With the modification described above, lowering of the image quality due to up and down breathing movement is suppressed to the minimum.

Further, the radiation imaging apparatus in accordance with the present invention may be modified such that the rotation control means controls such that the rotation driving of the set of the radiation source and the radiation detector by the rotation driving unit is ceased at the time of the radiation imaging operation, and such that the rotation driving is again begun after the radiation imaging operation has been performed. With the modification described above, the lowering of the image quality due to the motion artifact caused to occur by the rotation is prevented.

### BRIEF DESCRIPTION OF THE DRAWINGS

FIG. 1 is a perspective view showing an embodiment of the radiation imaging apparatus in accordance with the present invention,

FIG. 2 is a side view showing the embodiment of FIG. 1,

FIG. 3 is a block diagram showing the embodiment of FIG. 1,

FIG. 4 is a graph showing an example of a heartbeat signal detected by a heartbeat sensor in the embodiment of FIG. 1 and an example of a breathing signal detected by a breathing sensor in the embodiment of FIG. 1,

FIG. 5 is a graph showing an example of a state, in which driving of a rotation driving unit is performed or ceased by rotation control means shown in FIG. 3,

FIG. 6 is a graph showing a different example of a state, in which the driving of the rotation driving unit is performed or ceased by the rotation control means shown in FIG. 3,

FIG. 7 is an explanatory diagram showing a different example of a state, in which the driving of the rotation driving unit is performed or ceased by the rotation control means shown in FIG. 3,

FIG. 8 is a graph showing a different example of a state, in which the driving of the rotation driving unit is performed or ceased by the rotation control means shown in FIG. 3, and

FIG. 9 is a flow chart showing an embodiment of the radiation imaging method in accordance with the present invention.

### DESCRIPTION OF THE PREFERRED EMBODIMENTS

The present invention will hereinbelow be described in further detail with reference to the accompanying drawings.

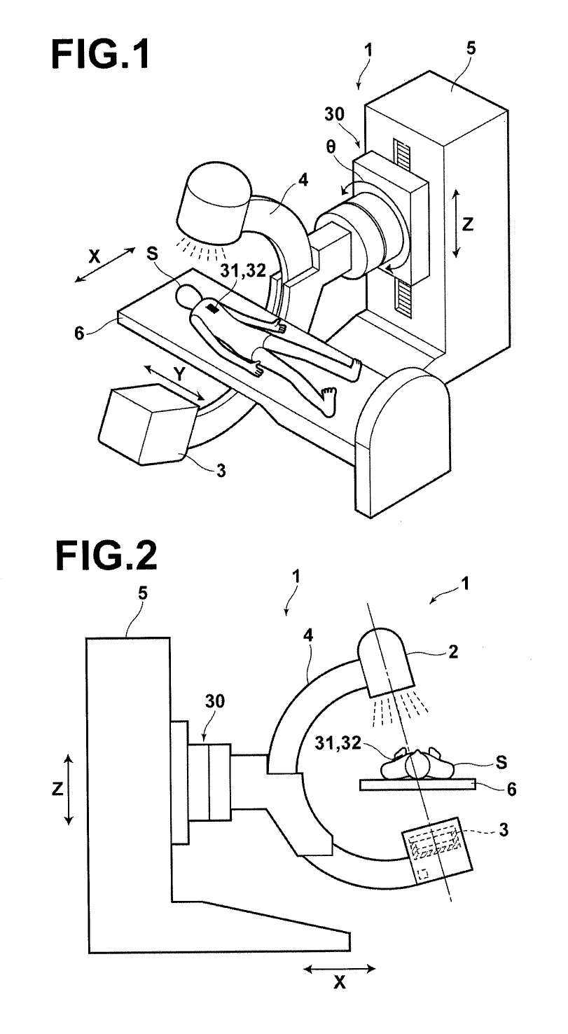

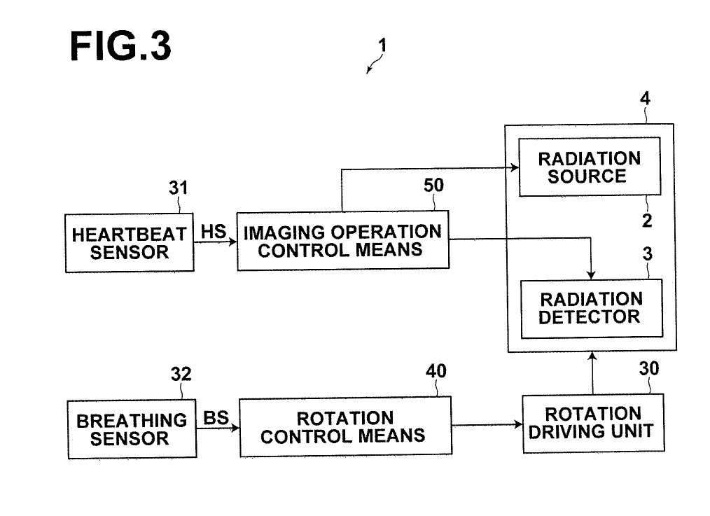

FIG. 1 is a perspective view showing an embodiment of the radiation imaging apparatus in accordance with the present invention. FIG. 2 is a side view showing the embodiment of FIG. 1. FIG. 3 is a block diagram showing the embodiment of FIG. 1. A radiation imaging apparatus 1 is a cone-beam CT scanner for performing imaging operations on an object from various different directions and thereby acquiring a plurality of radiation images to be used for the formation of a three-dimensional image. The radiation imaging apparatus 1 comprises a radiation source 2, a radiation detector 3, a C-arm 4, and a rotation driving unit. The radiation source 2 irradiates radiation toward an object S lying on a bed 6. The radiation detector 3 detects the radiation carrying image information of the object S as a radiation image at the time at which the radiation is irradiated from the radiation source 2 toward the object S.

The radiation source 2 and the radiation detector 3 are secured to opposite ends of the C-arm 4 so as to stand facing each other. The C-arm 4 is located on a support base 5 such that the C-arm 4 is capable of being moved by a rotation driving unit 30 in a direction indicated by the arrow θ and in a direction indicated by the arrow Z. Therefore, by the rotation of the C-arm 4, the set of the radiation source 2 and the radiation detector 3 is rotated around the object S for performing the radiation imaging operations. The rotation driving unit 30 rotates the set of the radiation source 2 and the radiation detector 3 at a velocity such that, for example, a period of time ranging from approximately several seconds to approximately several tens of seconds is required per turn.

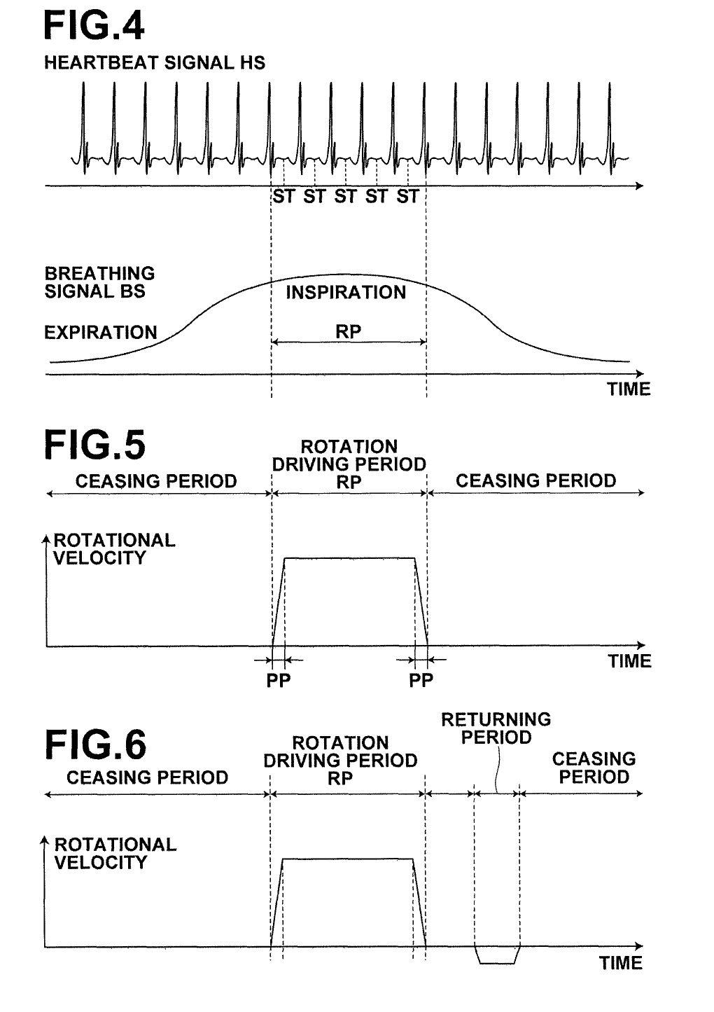

A heartbeat sensor 31 is an electrocardiograph for detecting the state of the heartbeat of the object S as a heartbeat signal HS. The heartbeat sensor 31 has a structure capable of being releasably fitted to the object S. The heartbeat signal HS is outputted from the heartbeat sensor 31 as the signal having a predetermined heartbeat period as illustrated in FIG. 4. Also, a breathing sensor 32 detects the state of the breathing of the object S as a breathing signal BS. The breathing sensor 32 has a structure capable of being releasably fitted to the object S. The breathing signal BS is outputted as a signal as illustrated in FIG. 4, which has a large signal value at the time of the inspiration, and which has a small signal value at the time of the expiration.

Rotation control means 40 illustrated in FIG. 3 controls the operation of the rotation driving unit 30 and thereby controls the position for the imaging operation performed by the set of the radiation source 2 and the radiation detector 3. In this embodiment, the rotation control means 40 controls the rotation driving unit 30, such that the rotation driving is begun at the time, at which the breathing signal BS having been detected by the breathing sensor 32 has come into a predetermined state. Specifically, the rotation control means 40 controls such that the operation of the rotation driving unit 30 is begun at the time, at which the breathing signal BS having been detected by the breathing sensor 32 represents at least 95% of the maximum breathing signal, and such that the driving of the rotation driving unit 30 is ceased at the time, at which the breathing signal BS represents a level lower than 95% of the maximum breathing signal.

Imaging operation control means 50 controls the radiation imaging operations performed by the set of the radiation source 2 and the radiation detector 3. In this embodiment, the imaging operation control means 50 controls such that the radiation imaging operations are performed at the time of a rotation driving period RP, during which the rotation driving unit 30 performs the rotation driving by being controlled by the rotation control means 40, and such that the radiation imaging operations are not performed at the time, at which the driving of the rotation driving unit 30 is being ceased. Also, the imaging operation control means 50 controls the operation of the set of the radiation source 2 and the radiation detector 3, such that the radiation imaging operation is performed at the time, at which the heart has come into a predetermined state, in accordance with the heartbeat signal HS having been detected by the heartbeat sensor 31. For example, the imaging operation control means 50 controls the operation of the set of the radiation source 2 and the radiation detector 3 such that, of a P wave, a Q wave, an R wave, an S wave, and a T wave, which are contained in the heartbeat signal HS having been detected by the heartbeat sensor 31, the timing synchronized with the R wave is taken as an imaging operation timing ST.

As described above, the driving and the ceasing of the rotation of the set of the radiation source 2 and the radiation detector 3, and the radiation imaging operations are controlled in accordance with the breathing signal BS and the heartbeat signal HS. In this manner, a plurality of the radiation images are acquired efficiently, such that the adverse effects of variation in state of the object due to the breathing and the heartbeat of the object S are suppressed to the minimum. Specifically, since the breathing is capable of being controlled artificially to a certain extent, in cases where ordinary radiation imaging operations are performed, the radiation imaging operations may be performed, while the breath is being stopped for a length of time of, for example, several seconds, and the adverse effects of the up and down movement due to the breathing are thereby capable of being suppressed. However, in the cases of the radiation imaging operations with the aforesaid cone-beam CT scanner, or the like, since an image operation time ranging from approximately several seconds to approximately several tens of seconds is required, it is not always possible to stop the breath until the radiation imaging operations are completed. In such cases, the radiation imaging operations for acquiring a necessary number of radiation images are performed over a plurality of times of breathing. However, if the imaging operations are merely performed only in the cases synchronized with the breathing and the heartbeat, while the set of the radiation source 2 and the radiation detector 3 are being rotated, many positions, at which the radiation imaging operations are not capable of being performed, will arise, and therefore the efficient radiation imaging operations will not be capable of being performed.

Therefore, in this embodiment, the rotation driving and the radiation imaging operations are turned on and off in accordance with the breathing and the heartbeat of the object S, and the plurality of the radiation images, in which the variation due to the up and down movement among the radiation images has been suppressed to the minimum, are thereby acquired efficiently without an excessive burden being imposed upon the object S. Accordingly, a three-dimensional reconstructed image is formed with a high accuracy such that a motion artifact is suppressed.

In the embodiment described above, the imaging operation control means 50 controls such that the radiation imaging operation is performed from the stage immediately after the driving of the rotation driving unit 30 has been begun. Alternatively, the radiation imaging operation may be begun after the driving of the rotation driving unit 30 has been begun and after the velocity of the set of the radiation source 2 and the radiation detector 3 has thus come up to a predetermined velocity. FIG. 5 is a graph showing an example of a state, in which the driving of the rotation driving unit 30 is performed or ceased by the rotation control means 40 shown in FIG. 3. Specifically, as illustrated in FIG. 5, the rotation driving unit 30 does not enable the set of the radiation source 2 and the radiation detector 3 to rotate at the predetermined velocity immediately after the rotation driving has been begun, but a preparatory period PP is required before the velocity of the set of the radiation source 2 and the radiation detector 3 come up to the predetermined velocity. In cases where the radiation imaging operation is begun after the velocity of the set of the radiation source 2 and the radiation detector 3 has come up to the predetermined velocity, the plurality of the radiation images are acquired under identical imaging operation conditions.

In cases where the preparatory period PP is short and is thus negligible, the radiation imaging operation may be performed only in the state, in which the velocity of the set of the radiation source 2 and the radiation detector 3 has come up to the predetermined velocity. However, it may often occur that a certain length of time is required as the preparatory period PP, and it is therefore desired to perform the radiation imaging operation also with respect to the preparatory period PP. In such cases, the imaging operation control means 50 may control such that the radiation imaging operation is performed with a timing synchronized with the heartbeat at the time, at which the set of the radiation source 2 and the radiation detector 3 takes a predetermined position for the radiation imaging operation (a predetermined rotation angle), within the preparatory period PP before the velocity of the set of the radiation source 2 and the radiation detector 3 comes up to the predetermined velocity.

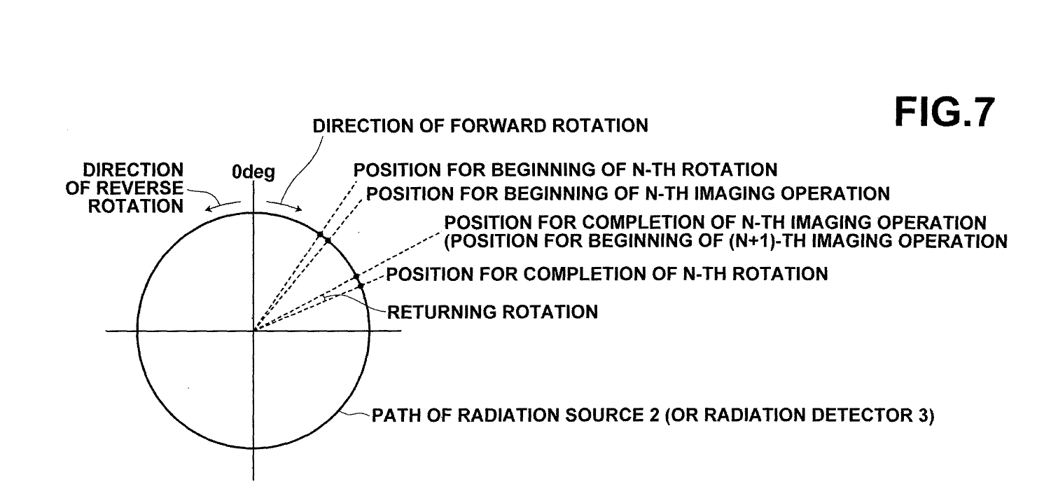

FIG. 6 is a graph showing a different example of a state, in which the driving of the rotation driving unit 30 is performed or ceased by the rotation control means 40 shown in FIG. 3. Also, FIG. 7 is an explanatory diagram showing a different example of a state, in which the driving of the rotation driving unit 30 is performed or ceased by the rotation control means 40 shown in FIG. 3. As another alternative, as illustrated in FIG. 6 and FIG. 7, the rotation control means 40 may drive the rotation driving unit 30, such that the position of the set of the radiation source 2 and the radiation detector 3 is returned to the position, which is taken at the time of the completion of the period of the imaging operation performed most recently, after the rotation driving period RP for the rotation driving performed most recently has been completed or before the next rotation driving period RP is begun. Specifically, the rotation driving unit 30 is rotated in the reverse direction by the angle corresponding to the rotation, which is performed during the preparatory period PP at the time of the initial movement of the rotation driving unit 30, and the rotation, which is performed during the preparatory period PP at the time of the final movement of the rotation driving unit 30, and the position of the set of the radiation source 2 and the radiation detector 3 is thereby returned. As a result, when the next radiation imaging operation is performed, the next radiation imaging operation is begun in the state, in which the set of the radiation source 2 and the radiation detector 3 is rotated at the predetermined velocity, from the position, at which the radiation imaging operation performed most recently has been completed (i.e., from the position, at which the rotation at the predetermined velocity has been completed at the stage of the radiation imaging operation performed most recently). Therefore, the problems are prevented from occurring in that a lack of a radiation image arises since the radiation imaging operation is not performed within the preparatory period PP.

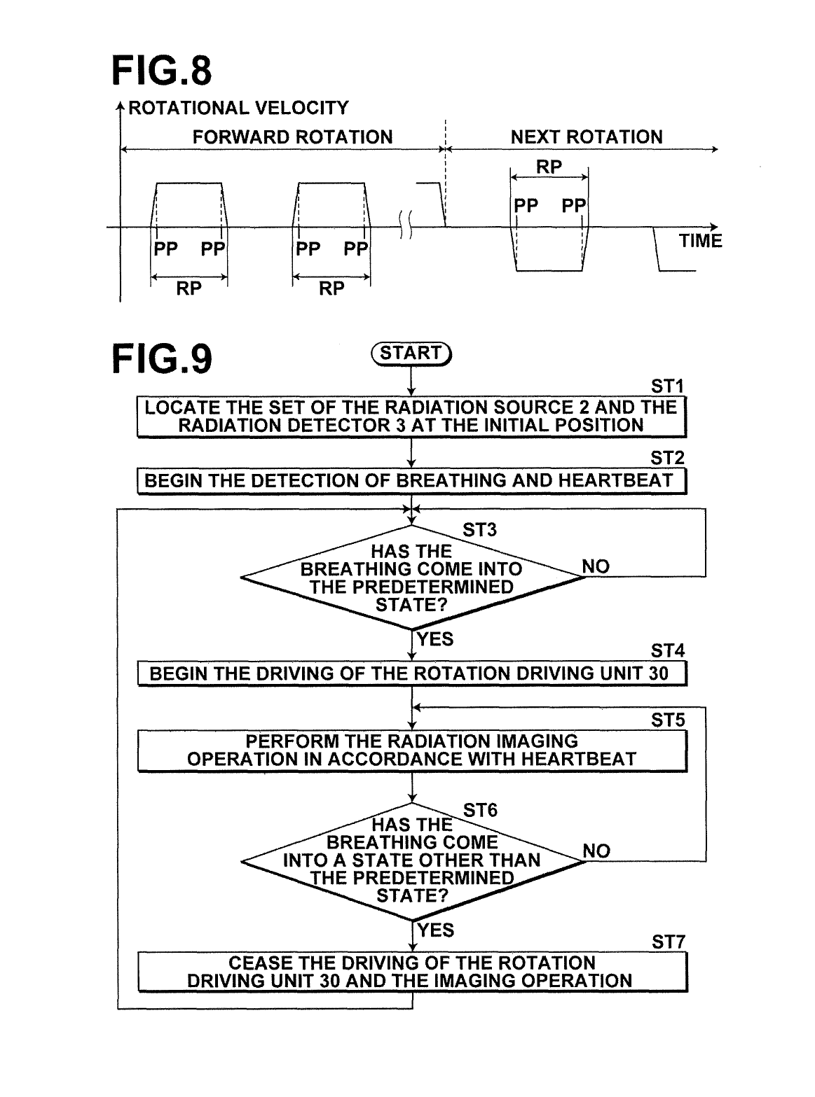

As a further alternative, in lieu of the position of the set of the radiation source 2 and the radiation detector 3 being returned at each of the stages, at which the operation of the rotation driving unit 30 is ceased, the set of the radiation source 2 and the radiation detector 3 may be rotated one turn, during which the radiation imaging operations are performed only in cases where the velocity of the set of the radiation source 2 and the radiation detector 3 has come up to the predetermined velocity. Thereafter, the rotation driving unit 30 may be rotated in the direction, which is identical with the direction of rotation performed one turn as described above, or in the reverse direction, and the radiation imaging operation may then be performed with respect to the position for the radiation imaging operation within the preparatory period PP. FIG. 8 is a graph showing a different example of a state, in which the driving of the rotation driving unit 30 is performed or ceased by the rotation control means 40 shown in FIG. 3. Specifically, as illustrated in FIG. 7 and FIG. 8, the imaging operation control means 50 controls such that the radiation imaging operation is begun after the driving of the rotation driving unit 30 has been begun and after the velocity of the set of the radiation source 2 and the radiation detector 3 has thus come up to the predetermined velocity. Also, the rotation control means 40 controls such that the set of the radiation source 2 and the radiation detector 3 is rotated one turn. Thereafter, the rotation control means 40 controls the rotation driving unit 30 such that the set of the radiation source 2 and the radiation detector 3 is rotated in the forward direction or in the reverse direction in order for the radiation imaging operation to be performed with respect to the position corresponding to the preparatory period PP. In such cases, the position for the imaging operation, which position corresponds to the preparatory period PP, has been detected at the time of the rotation driving performed most recently, and the imaging operation control means 50 controls such that the radiation imaging operation in accordance with the breathing and the heartbeat is performed with respect to the thus detected position for the imaging operation. In such cases, the problems are prevented from occurring in that a lack of a radiation image arises since the radiation imaging operation is not performed within the preparatory period PP.

FIG. 9 is a flow chart showing an embodiment of the radiation imaging method in accordance with the present invention. The embodiment of the radiation imaging method in accordance with the present invention will hereinbelow be described with reference to FIG. 1 through FIG. 9. Firstly, in the state in which the object S lies on the bed 6, the heartbeat sensor 31 and the breathing sensor 32 are fitted to the object S. Thereafter, in a step ST1, the set of the radiation source 2 and the radiation detector 3 is located at the initial position. Also, in a step ST2, the detection of the heartbeat signal HS by the heartbeat sensor 31 and the detection of the breathing signal BS by the breathing sensor 32 are begun.

Thereafter, in a step ST3, a judgment is made by the rotation control means 40 as to whether the breathing signal BS has or has not come into the predetermined state (in which the breathing signal BS represents at least 95% of the maximum inspiration). (Reference may be made to FIG. 4.) In a step ST4, in cases where it has been judged that the breathing signal BS has come into the predetermined state, the driving of the rotation driving unit 30 is begun, and the set of the radiation source 2 and the radiation detector 3 is rotated around the object S. At this time, in a step ST5 and through a step ST6, the imaging operation control means 50 takes a predetermined temporal phase of the heartbeat signal HS as each of imaging operation timings ST, ST, . . . . The rotation of the set of the radiation source 2 and the radiation detector 3 and a plurality of the radiation imaging operations are performed with the imaging operation timings ST, ST, . . . . (Reference may be made to FIG. 4 and FIG. 5.)

In cases where it has been judged by the rotation control means 40 in the step ST6 that the breathing signal BS has come into a state other than the predetermined state (in which the breathing signal BS represents at least 95% of the maximum inspiration), in a step ST7, the rotation driving of the set of the radiation source 2 and the radiation detector 3 by the rotation driving unit 30 is ceased, and the radiation imaging operation is ceased. (Reference may be made to FIG. 5.) Thereafter, the judgment is made by the rotation control means 40 as to whether the breathing signal BS has or has not come into the predetermined state (in which the breathing signal BS represents at least 95% of the maximum inspiration). Also, in cases where it has been judged that the breathing signal BS has come into the predetermined state, the radiation imaging operations are performed. In the step ST3 through the step ST7, the aforesaid operations for making the judgment and for performing the radiation imaging operations are iterated. In such cases, as illustrated in FIG. 6 and FIG. 7, after the driving of the rotation driving unit 30 has been ceased, the position of the set of the radiation source 2 and the radiation detector 3 may be returned by an angle corresponding to the preparatory period PP. Alternatively, as illustrated in FIG. 7 and FIG. 8, after one turn of the set of the radiation source 2 and the radiation detector 3 has been completed, the set of the radiation source 2 and the radiation detector 3 may be rotated in the forward direction or in the reverse direction, and the radiation imaging operation may then be performed with respect to the position for the imaging operation, which position corresponds to the preparatory period PP.

With the embodiment described above, the plurality of the radiation images of the object S are acquired by iterating the steps of:

beginning the driving of the rotation driving unit 30 at the time at which the breathing signal BS having been detected by the breathing sensor 32 has come into the predetermined state,

performing the radiation imaging operation with the set of the radiation source 2 and the radiation detector 3 at the time, at which the heart has come into the predetermined state, in accordance with the heartbeat signal HS having been detected by the heartbeat sensor 31 within the rotation driving period, during which the rotation driving unit 30 performs the rotation driving, and

ceasing the rotation driving of the rotation driving unit 30 at the time at which the breathing of the object S has come into a state other than the predetermined state.

Thus, with the embodiment described above, in cases where the set of the radiation source 2 and the radiation detector 3 is rotated around the object S and performs the radiation imaging operations in accordance with the breathing and the heartbeat of the object S, the rotation driving is ceased with respect to the period, during which the breathing and the imaging operation timing do not match with each other, and during which the radiation imaging operation is not capable of being performed. Thus the problems are prevented from occurring in that the rotation driving alone is performed without the set of the radiation source 2 and the radiation detector 3 performing the radiation imaging operation. Therefore, the plurality of the radiation images are acquired efficiently, such that the adverse effects of the variation in state of the object S due to the breathing and the heartbeat of the object S are suppressed to the minimum.

As illustrated in FIG. 5, the radiation imaging apparatus 1 in accordance with the present invention may be modified such that the rotation driving unit 30 rotates the set of the radiation source 2 and the radiation detector 3 at the predetermined velocity, and

the imaging operation control means 50 controls the operation of the set of the radiation source 2 and the radiation detector 3, such that the radiation imaging operation is performed after the set of the radiation source 2 and the radiation detector 3 has been driven for rotation from the state, in which the set of the radiation source 2 and the radiation detector 3 stops, and after the velocity of the set of the radiation source 2 and the radiation detector 3 has thus come up to the predetermined velocity.

At the time at which the rotation of the set of the radiation source 2 and the radiation detector 3 is begun, a certain length of time is required due to inertia force before the velocity of the set of the radiation source 2 and the radiation detector 3 comes up to the predetermined velocity. With the modification described above, wherein the imaging operation control means 50 controls the operation of the set of the radiation source 2 and the radiation detector 3, such that the radiation imaging operation is begun after the velocity of the set of the radiation source 2 and the radiation detector 3 has come up to the predetermined velocity, a difference in imaging operation conditions among the plurality of the radiation imaging operations is suppressed to the minimum, and the image quality of the three-dimensional image reconstructed from the plurality of the radiation images is prevented from becoming bad.

Also, as illustrated in FIG. 6 and FIG. 7, the radiation imaging apparatus 1 in accordance with the present invention may be modified such that the rotation control means 40 has the function for returning the position of the set of the radiation source 2 and the radiation detector 3 in the direction, which is reverse to the direction of the rotation of the set of the radiation source 2 and the radiation detector 3 at the time of the radiation imaging operation, after the radiation imaging operation performed most recently has been completed or before the next radiation imaging operation is begun, such that the rotation driving of the set of the radiation source 2 and the radiation detector 3 is begun at the predetermined velocity from the position, at which the rotation driving of the rotation driving unit 30 has been ceased at the stage of the radiation imaging operation performed most recently. With the modification described above, the problems are prevented from occurring in that a position, at which the radiation imaging operation is not performed, arises with respect to the period between the stage, at which the rotation of the set of the radiation source 2 and the radiation detector 3 is begun, and the stage, at which the velocity of the set of the radiation source 2 and the radiation detector 3 comes up to the predetermined velocity.

Further, the radiation imaging apparatus 1 in accordance with the present invention may be modified such that the imaging operation control means 50 controls such that the radiation imaging operation is performed at the time, at which the set of the radiation source 2 and the radiation detector 3 takes the predetermined angular position, during the period between the state, in which the set of the radiation source 2 and the radiation detector 3 stops, and the stage, at which the velocity of the set of the radiation source 2 and the radiation detector 3 comes up to the predetermined velocity. With the modification described above, the problems are prevented from occurring in that a position, at which the radiation imaging operation is not performed, arises with respect to the period between the stage, at which the rotation of the set of the radiation source 2 and the radiation detector 3 is begun, and the stage, at which the velocity of the set of the radiation source 2 and the radiation detector 3 comes up to the predetermined velocity.

Furthermore, as illustrated in FIG. 7 and FIG. 8, the radiation imaging apparatus 1 in accordance with the present invention may be modified such that the rotation control means 40 controls the rotation driving unit 30 such that the set of the radiation source 2 and the radiation detector 3 is rotated the plurality of turns, and

the imaging operation control means 50 controls the operation of the set of the radiation source 2 and the radiation detector 3, such that the radiation imaging operation is performed with respect to the angular position taken by the set of the radiation source 2 and the radiation detector 3 during the period between the state, in which the set of the radiation source 2 and the radiation detector 3 stops at the time of the predetermined turn, and the stage, at which the velocity of the set of the radiation source 2 and the radiation detector 3 comes up to the predetermined velocity at the time of the predetermined turn, the radiation imaging operation with respect to the angular position being performed at the time of the next turn.

With the modification described above, the problems are prevented from occurring in that a position, at which the radiation imaging operation is not performed, arises with respect to the period between the stage, at which the rotation of the set of the radiation source 2 and the radiation detector 3 is begun, and the stage, at which the velocity of the set of the radiation source 2 and the radiation detector 3 comes up to the predetermined velocity.

Also, the radiation imaging apparatus 1 in accordance with the present invention may be modified such that the rotation control means 40 begins the driving of the rotation driving unit 30 at the time, at which the breathing signal BS represents at least 95% of the maximum inspiration, and such that the rotation control means 40 ceases the driving of the rotation driving unit 30 at the time, at which the breathing signal BS represents a level lower than 95% of the maximum inspiration. With the modification described above, lowering of the image quality due to the up and down breathing movement is suppressed to the minimum.

The radiation imaging apparatus and the radiation imaging method in accordance with the present invention may be embodied in various other ways. For example, FIG. 4 illustrates the cases where the rotation driving of the rotation driving unit 30 is begun at the time, at which the breathing signal BS represents at least 95% of the maximum inspiration. Alternatively, since the breathing is capable of being controlled artificially to a certain extent, voice outputting or display outputting for navigation may be performed, and the beginning or the completion of the rotation driving may be controlled in accordance with a specified timing and the breathing signal BS.

Also, in the embodiment described above, the radiation imaging operation is performed with the predetermined imaging operation timing under the condition in which the set of the radiation source 2 and the radiation detector 3 is being rotated. In cases where it is assumed that the X-ray irradiation is performed within a sufficiently short period of time with respect to the rotational velocity (for example, within an X-ray irradiation time of 1 ms in cases where the rotational velocity is 360 deg/30 sec), the motion artifact due to the rotation becomes as small as a negligible extent.

From the view point of the motion artifact, the rotation control means 40 may rotate or stop the set of the radiation source 2 and the radiation detector 3 in accordance with the breathing signal BS and may control the rotation driving unit 30 such that the rotation driving of the set of the radiation source 2 and the radiation detector 3 is ceased at the time of the radiation imaging operation. Specifically, in such cases, the radiation imaging operation is performed in the state in which the set of the radiation source 2 and the radiation detector 3 stops. Also, after the radiation imaging operation has been completed, the rotation control means 40 rotates the set of the radiation source 2 and the radiation detector 3 to the position for the next imaging operation (for example, by a specified angle of 1 deg) or to the position corresponding to the next imaging operation timing. The operations described above are iterated within the aforesaid rotation driving period RP. In such cases, the lowering of the image quality due to the motion artifact caused to occur by the rotation is prevented.

What is claimed is:

1. A radiation imaging apparatus, comprising:
i) a radiation source for irradiating radiation to an object,
ii) a radiation detector for detecting the radiation carrying image information of the object at the time at which the radiation is irradiated from the radiation source to the object,
iii) a rotation driving unit for rotating the set of the radiation source and the radiation detector around the object,
iv) a breathing sensor for detecting a state of breathing of the object as a breathing signal,
v) a heartbeat sensor for detecting a state of heartbeat of the object as a heartbeat signal,
vi) rotation control means for controlling such that the driving of the rotation driving unit is begun at the time at which the breathing signal having been detected by the breathing sensor has come into a predetermined state, and such that the driving of the rotation driving unit is ceased at the time at which the breathing of the object has come into a state other than the predetermined state, and
vii) imaging operation control means for controlling the operation of the set of the radiation source and the radiation detector, such that a radiation imaging operation is performed at the time, at which the heart has come into a predetermined state, in accordance with the heartbeat signal having been detected by the heartbeat sensor within a rotation driving period, during which the rotation driving unit performs the rotation driving by being controlled by the rotation control means.

2. A radiation imaging apparatus as defined in claim 1 wherein the rotation driving unit rotates the set of the radiation source and the radiation detector at a predetermined velocity, and
the imaging operation control means controls the operation of the set of the radiation source and the radiation detector, such that the radiation imaging operation is performed after the set of the radiation source and the radiation detector has been driven for rotation from the state, in which the set of the radiation source and the radiation detector stops, and after the velocity of the set of the radiation source and the radiation detector has thus come up to the predetermined velocity.

3. A radiation imaging apparatus as defined in claim 1 wherein the rotation control means has a function for returning the position of the set of the radiation source and the radiation detector in the direction, which is reverse to the direction of the rotation of the set of the radiation source and the radiation detector at the time of the radiation imaging operation, after a radiation imaging operation performed most recently has been completed or before a next radiation imaging operation is begun, such that the rotation driving of the set of the radiation source and the radiation detector is begun at a predetermined velocity from a predetermined position.

4. A radiation imaging apparatus as defined in claim 3 wherein the rotation control means has the function for returning the position of the set of the radiation source and the radiation detector, such that the rotation driving of the set of the radiation source and the radiation detector is begun at the predetermined velocity from the position, at which the rotation driving of the rotation driving unit has been ceased at the stage of the radiation imaging operation performed most recently.

5. A radiation imaging apparatus as defined in claim 2 wherein the imaging operation control means controls such that the radiation imaging operation is performed at the time, at which the set of the radiation source and the radiation detector takes a predetermined angular position, during the period between the state, in which the set of the radiation source and the radiation detector stops, and the stage, at which the velocity of the set of the radiation source and the radiation detector comes up to the predetermined velocity.

6. A radiation imaging apparatus as defined in claim 2 wherein the rotation control means controls the rotation driving unit such that the set of the radiation source and the radiation detector is rotated a plurality of turns, and
the imaging operation control means controls the operation of the set of the radiation source and the radiation detector, such that the radiation imaging operation is performed with respect to an angular position taken by the set of the radiation source and the radiation detector during the period between the state, in which the set of the radiation source and the radiation detector stops at the time of a predetermined turn, and the stage, at which the velocity of the set of the radiation source and the radiation detector comes up to the predetermined velocity at the time of the predetermined turn, the radiation imaging operation with respect to the angular position being performed at the time of the next turn.

7. A radiation imaging apparatus as defined in claim 1 wherein the rotation control means controls such that the rotation driving of the set of the radiation source and the radiation detector by the rotation driving unit is ceased at the time of the radiation imaging operation, and such that the rotation driving is again begun after the radiation imaging operation has been performed.

8. A radiation imaging apparatus as defined in claim 1 wherein the rotation control means begins the driving of the rotation driving unit at the time, at which the breathing signal represents at least 95% of the maximum inspiration, and such that the rotation control means ceases the driving of the rotation driving unit at the time, at which the breathing signal represents a level lower than 95% of the maximum inspiration.

9. A radiation imaging method, utilizing:
i) a radiation source for irradiating radiation to an object,
ii) a radiation detector for detecting the radiation carrying image information of the object at the time at which the radiation is irradiated from the radiation source to the object,
iii) a rotation driving unit for rotating the set of the radiation source and the radiation detector around the object,
iv) a breathing sensor for detecting a state of breathing of the object as a breathing signal, and
v) a heartbeat sensor for detecting a state of heartbeat of the object as a heartbeat signal,
wherein a plurality of radiation images of the object are acquired by iterating the steps of:
beginning the driving of the rotation driving unit at the time at which the breathing signal having been detected by the breathing sensor has come into a predetermined state,
performing a radiation imaging operation with the set of the radiation source and the radiation detector at the time, at which the heart has come into a predetermined state, in accordance with the heartbeat signal having been detected by the heartbeat sensor within a rotation driving period, during which the rotation driving unit performs the rotation driving, and
ceasing the rotation driving of the rotation driving unit at the time at which the breathing of the object has come into a state other than the predetermined state.
